(19) United States
(12) Patent Application Publication
(10) Pub. No.: US 2023/0046620 A1
(43) Pub. Date: Feb. 16, 2023

(54) LONG-WEARING COSMETIC COMPOSITION COMPRISING EXPANDED PERLITE

(71) Applicant: L'OREAL, PARIS (FR)

(72) Inventor: Christopher PANG, Nanuet, NY (US)

(73) Assignee: L'OREAL, PARIS (FR)

(21) Appl. No.: 17/387,196

(22) Filed: Jul. 28, 2021

(51) Int. Cl. A61K 8/25; A61K 8/891; A61K 8/88; A61K 8/92; A61Q 1/06

(52) U.S. Cl. CPC A61K 8/25; A61K 8/891; A61K 8/88; A61K 8/92; A61Q 1/06; A61K 2800/31; A61K 2800/412; A61K 2800/43; A61K 2800/594

(57) ABSTRACT

The present invention relates to anhydrous compositions including at least one silicone film former; from about 10% by weight to about 20% by weight of expanded perlite having an average particle size of less than about 50 microns; at least one silicone elastomer; and at least one volatile solvent.

### FIELD OF THE INVENTION

The present invention relates to a cosmetic composition and method for making up and/or enhancing the appearance of a keratinous substrate, and in particular, compositions including silicone film-formers; expanded perlite; silicone elastomer; and volatile solvent.

### BACKGROUND OF THE INVENTION

Cosmetic compositions used to make up or enhance the appearance of a user's skin are often required to impart various properties such as long wear, transfer resistance and comfort. However, the formulation of cosmetic products that can deliver these properties at the same time can pose some challenges. For example, cosmetic compositions using traditional ingredients known to impart long wear, such as silicone resins, are very drying. In addition, they cause discomfort and flaking during the use. In order to overcome these problems, oils, such as silicone oils are generally employed. While the utilization of silicone oils in cosmetics is popular, one drawback associated with their use is that they tend to shine and are tacky, which are not always desired effects for the finished products. Furthermore, for products in which it is also desirable to reduce gloss (increase mattity), attempting to reduce gloss often results in reduction of long-wear performance.

Therefore, it is an object of the present invention to provide a composition and method for making up skin in a manner which delivers transfer resistance and long wear properties, as well as superior comfort, non-tacky feel and nonglossy (matte) appearance.

It has been surprisingly discovered that the using relatively high concentrations of relatively small size particles of expanded perlite in a system with long-wear polymers, silicone elastomer, and volatile solvent, provides compositions characterized by matte appearance, non-tacky feel and superior comfort, transfer resistance, long wear when applied onto a keratinous substrate.

### BRIEF SUMMARY OF THE INVENTION

According to one embodiment of the present invention, there is provided an anhydrous composition which is long wearing and transfer resistant, while at the same time provides superior comfort, non-tacky feel and looks matte. The composition includes at least one silicone film former (such as silicone resin and/or polyorganosiloxane copolymer); from about 10% by weight to about 20% by weight of expanded perlite having an average particle size of less than about 50 microns; at least one silicone elastomer; and at least one volatile solvent. The compositions are anhydrous.

According to a preferred embodiment, the composition further includes at least one colorant and/or at least one wax, and/or at least one non-volatile oil.

In certain embodiments, these compositions are characterized by having a ratio of the concentration by weight of the expanded perlite to the concentration by weight of the at least one colorant is from about 1:1 to about 4:1. In certain other embodiments, these compositions are characterized by having a ratio of the concentration by weight of the expanded perlite to a total concentration by weight of silicone film-forming polymer (e.g., silicone resin and/or polyorganosiloxane copolymer) is from about 0.5:1 to about 1.1:1. In certain other embodiments, both of these ratios are satisfied.

As per this invention, the inventive compositions are related but not limited to liquid compositions, such as liquid lipsticks, liners, foundations, mascaras, eyeshadows, skin care compositions, sunscreens, skin repellants, deodorants, nail composition.

In another embodiment, the invention is a method of making up skin involving applying onto the skin the above disclosed compositions, as well as a method of making the inventive composition.

As per another embodiment, this invention relates to a system of cosmetic compositions comprising (a first) composition as described herein and (a second) top coat composition.

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% to 15% of the indicated number (e.g. “about 10%” means 8.5% to 11.5% such as 9% to 11% and “about 2%” means from 1.7% to 2.3 such as from 1.8% to 2.2%).

“Film former” or “film forming agent” or “film forming polymer” or “film forming resin” as used herein mean a polymer or resin that leaves a film on the substrate to which it is applied, for example, after a solvent accompanying the film former has evaporated, absorbed into and/or dissipated on the substrate. A “silicone film former” is a film former that includes at least one silicone (a silicone atom bonded directly to an oxygen atom and also to a carbon atom such as in an organic moiety).

“Transfer resistance” as used herein refers to the quality exhibited by compositions that are not readily removed by contact with another material, such as, for example, a glass, an item of clothing or the skin, for example, when eating or drinking. Transfer resistance may be evaluated by any method known in the art for evaluating such. For example, transfer resistance of a composition may be evaluated by a “kiss” test. The “kiss” test may involve application of the composition to human keratin material such as hair, skin or lips followed by rubbing a material, for example, a sheet of paper, against the hair, skin or lips after expiration of a certain amount of time following application, such as 2 minutes after application. Similarly, transfer resistance of a composition may be evaluated by the amount of product transferred from a wearer to any other substrate, such as transfer from the hair, skin or lips of an individual to a collar when putting on clothing after the expiration of a certain amount of time following application of the composition to the hair, skin or lips. The amount of composition transferred to the substrate (e.g., collar, or paper) may then be evaluated and compared. For example, a composition may be transfer resistant if a majority of the product is left on the wearer's hair, skin or lips. Further, the amount transferred may be compared with that transferred by other compositions, such as commercially available compositions. In a preferred embodiment of the present invention, little or no composition is transferred to the substrate from the hair, skin or lips.

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions. For lip compositions, “long wear” typically means the composition remains on the lips at least about 4 hours up to about 24 hours, and retains rich color even after eating.

“Liquid” or “liquid cosmetic” or “liquid lipstick” or “liquoid composition” means a composition having a fixed volume, flows to cover the bottom and assumes the shape of the portion of the container it fills and is slightly compressible.

“Tackiness” as used herein refers to the adhesion between two substances. For example, the more tackiness there is between two substances, the more adhesion there is between the substances. To quantify “tackiness,” it is useful to determine the “work of adhesion” as defined by IUPAC associated with the two substances. Generally speaking, the work of adhesion measures the amount of work necessary to separate two substances. Thus, the greater the work of adhesion associated with two substances, the greater the adhesion there is between the substances, meaning the greater the tackiness is between the two substances.

Work of adhesion and, thus, tackiness, can be quantified using acceptable techniques and methods generally used to measure adhesion and the one far along described.

“Substituted” as used herein, means comprising at least one substituent. Non-limiting examples of substituents include atoms, such as oxygen atoms and nitrogen atoms, as well as functional groups, such as amine groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphategroups, siloxane groups, and polysiloxane groups. The substituent(s) may be further substituted.

“Substantially free” as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. In certain embodiments, substantially free means less than about 2% of the identified ingredient, such as less than about 1%, such as less than about 0.5% based on the composition as a whole. The term “anhydrous” means substantially free of water.

Numerical ranges are inclusive of endpoints and meant to include all combinations and sub-combinations. For example, from about 5%, 10% or 15% to about 20%, 50% or 60% may refer to about 5% to about 20%, about 5% to about 50%, about 5% to about 60%, about 10% to about 20%, about 10% to about 50%, about 10% to about 60%, about 15% to about 20%, about 15% to about 50%, or about 15% to about 60%. As used herein a range of ratios is meant to include every specific ratio within, and combination of subranges between the given ranges.

The compositions and methods of the present invention can comprise, consist of, or consist essentially of the essential elements and limitations of the invention described herein, as well as any additional or optional ingredients, components, or limitations described herein or otherwise useful.

Silicone Resins

The cosmetic compositions of the present invention include at least one silicone film-former such as a silicone resin. Examples of silicone resins are described in U.S. Pat. Nos. 5,505,937, 5,911,974, 5,965,112, 5,985,298, 6,074,654, 6,780,422, 6,908,621, the disclosures of which are hereby incorporated by references.

According to certain embodiments the composition includes a silicone resin selected from a siloxysilicate resin, a silsequioxane resin, and combinations thereof. One non-limiting example of a siloxysilicate in accordance with the present invention is trimethylsiloxysilicate, which may be represented by the following formula:

[(CH₃)₃SiO]ₓ(SiO_{4/2})_{y}

wherein x and y may, for example, range from 50 to 80. Such siloxysilicates are commercially available from General Electric, Dow Corning, Wacker, Milliken, Siltech, Grant Industries, Momentive and Shin-Etsu Silicones under the tradename Resin MQ®.

According to another embodiment of this invention, the compositions may contain silsesquioxane resins, including comprise at least one polypropyl silsesquioxane film forming resin.

Silsesquioxane resins are a specific form of silicone resin. Silicone resin nomenclature is known in the art as “MDTQ” nomenclature, whereby a silicone resin is described according to the various monomeric siloxane units which make up the polymer. Each letter of “MDTQ” denotes a different type of unit. When the film forming resin is made up predominantly of tri-functional units (or T units), it is generally called a silsesquioxane resin, which is described, for example in US 2006/0292096, herein incorporated by reference.

Examples of silsesquioxane resins that may be used in the present invention are alkyl silsesquioxane resins that are silsesquioxane homopolymers and/or copolymers having an average siloxane unit of the general formula R¹ₙSiO_{(4-n)/2}, wherein each R1 is a propyl group, wherein more than 80 mole % of R1 represent a C3-C10 alkyl group, n is a value of from 1.0 to 1.4, and more than 60 mole % of the copolymer comprises R¹SiO_{3/2} units. As each R1 is a propyl group these polymers are called polypropylsilsesquioxane resins or “t-propyl” silsesquioxane resins. These resins and methods of making them are described, for example in U.S. Pat. No. 8,586,013, 2012/0301415, 2007/0093619, and 2006/0292096, all of which are herein incorporated by reference.

A non-limiting example of a polypropylsilsesquioxane resin suitable for use in the present invention is commercially available from Dow Corning as Dow Corning 670 Fluid or Dow Corning 680 Fluid. These Dow Corning resins have a general formula of RₙSiO_{(4-n)/2} wherein R is independently chosen from a hydrogen atom and a monovalent hydrocarbon group comprising 3 carbon atoms, wherein more than 80 mole % of R are propyl groups, n is a value from 1.0 to 1.4, more than 60 mole % of the copolymer comprises RSiO_{3/2} units, and having a hydroxyl or alkoxy content from 0.2 to 10% by weight, for example between 1 and 4% by weight, preferably between 5 and 10% by weight, and more preferably between 6 and 8% by weight. Preferably, the polypropylsilsesquioxane resin has a molecular weight from about 5000 to about 30,000 and a Tg from about −5° C. to about 5° C.

Another embodiment of this invention exemplifies the composition containing at least one siloxysilicate resin, at least one silsesquioxane resin and/or mixture thereof.

The at least one silicone resin is generally present in the cosmetic composition of the present invention in an amount ranging from about 1% to about 25% such as 1%, 2%, 4% or 5% to about 10%, 15%, 20% or 25%, all weights being based on the weight of the composition as a whole.

Polyorganosiloxane Copolymer

Other silicone film-formers suitable for use in compositions of the present invention include polyorganosiloxane-containing polymers. The polyorganosiloxane-containing polymer useful herein is a polymer (homopolymer or copolymer) having at least one moiety which contains: at least one polyorganosiloxane group consisting of 1 to about 1000 organosiloxane units in the chain of the moiety or in the form of a graft, and at least two groups capable of establishing hydrogen interactions. Non-limiting examples of polyorganosiloxane-containing polymers are disclosed, for example in U.S. Pat. No. 8,945,525, the disclosure of which is hereby incorporated by reference.

Additional polyorganosiloxane-containing polymers which may be used in the composition of the invention include those described in documents U.S. Pat. No. 5,874,069, U.S. Pat. Nos. 5,919,441, 6,051,216, and 5,981,680, the entire contents of which are hereby incorporated by reference.

A preferred polyorganosiloxane-containing polymer for use in the present invention contain at least one moiety chosen from formula (III):

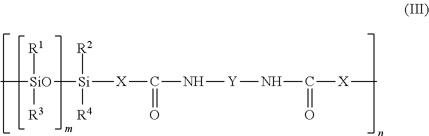

and formula (IV)

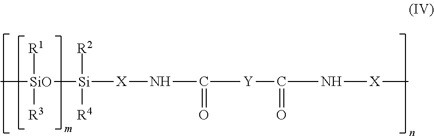

in which:
(a) R¹, R², R³ and R⁴ are the same or different and may be selected from the group consisting of methyl, ethyl, propyl, isopropyl, a siloxane chain, and phenyl;
(b) X is a linear or branched chain alkylene having 1-30 carbons;
(c) Y is selected from the group consisting of linear or branched chain alkylenes having 1-40 carbons;
(d) m is a number between 1 and 700;
(e) n is a number between 1 and 500.

Particularly preferred polyorganosiloxane-containing polymers useful herein are commercially available from Dow Corning under the tradenames DC 8178® and DC 8179®, which are known under the INCI denomination of Nylon-611/Dimethicone Copolymer.

The at least one polyorganosiloxane-containing polymer is generally present in the cosmetic composition of the present invention in an amount ranging from about 1% to about 25% such as 1%, 2% 4% or 7% to about 10%, 20% or 25%, all weights being based on the weight of the composition as a whole.

Silicone Elastomer

The composition according to the invention also includes at least one silicone elastomer (silicone crosspolymer). Such silicone elastomers are generally not qualify as “silicone film-formers,” as they tend to form particulates that do not form films.

In a preferred embodiment, the composition comprises a non-emulsifying silicon elastomer. The non-emulsifying silicon elastomer may be in the form of a gel or a powder.

The “organopolysiloxane elastomer” or “silicon elastomer” or “silicone crosspolymer” thickens the composition, adds the cushiony (spongy) effect and to improves the application of the finished product. Also, it provides a very soft feel and mattifying effect after the application, which is especially advantageous for skin products.

The term “non-emulsifying” defines organopolysiloxane elastomers that do not contain in any hydrophilic chains, and in particular polyoxyalkylene (especially polyoxyethylene or polyoxypropylene) or polyglyceryl units. Thus, according to one particular embodiment of the invention, the composition comprises an organopolysiloxane elastomer that is free of polyoxyalkylene units and polyglyceryl units.

The non-emulsifying elastomers are described in U.S. Pat. No. 8,637,057, the disclosure of which is hereby incorporated by reference.

The non-emulsifying elastomers particularly useful in this invention include but not limit those sold under the names KSG-6, KSG-15, KSG-16, KSG-18, KSG-41, KSG-42, KSG-43 and KSG-44 by the company Shin-Etsu, DC 9040, DC 9041, DC 9509, DC 9505 and DC 9506, by the company Dow Corning, and SFE 839 by the company General Electric.

In an embodiment, the organopolysiloxane elastomer particles are conveyed in the form of a gel formed from an elastomeric organopolysiloxane included in at least one hydrocarbon-based oil and/or one silicone oil. In these gels, the organopolysiloxane particles are often non-spherical particles.

Not limited examples of silicone elastomers useful in this invention are dimethicone crosspolymer gels (available as blends of dimethicone crosspolymers in solvents) having viscosity values from about 150 and to about 700 mm²/s, from about 200 to about 650 mm²/s and from about 300 to about 600 mm²/s.

Particularly useful for this invention may be blends of high molecular weight silicone elastomers in volatile solvents, such as silicone oils, hydrocarbon oils and mixtures thereof, as per definition disclosed far along.

The specific but not limiting examples of silicone elastomeric gels applicable in this invention are represented by DC EL-8040 ID (INCI name Isododecane (and) Dimethicone Crosspolymer) and DC EL-9140 DM (INCI name: Dimethicone (and) Dimethicone Crosspolymer) and DOWSIL EL-9240 Silicone Elastomer Blend (INCI name Dimethicone (and) Dimethicone Crosspolymer), supplied by Dow Corning.

Non-limiting examples of silicone elastomers and their synthesis are disclosed, for example in U.S. Pat. No. 8,637,057 and US/20150174048, all of which are herein incorporated by reference.

The at least one silicone elastomer is generally present in the cosmetic composition of the present invention in an amount ranging from about 1% to about 25% such as 1%, 2% or 4% to about 5%, 10%, 15% or 20%, all weights being based on the weight of the composition as a whole.

Volatile Solvent

The compositions of the invention include at least one volatile solvent.

In certain embodiments, the expression “volatile solvent” means any non-aqueous compound capable of evaporating on contact with the skin or the lips in less than one hour at room temperature and atmospheric pressure. In certain other embodiments “volatile solvent” means any non-aqueous compound having a flash point of less than about 120° C., such as less than about 100° C., such as from about 40° C. to about 100° C.

Examples of suitable volatile solvents include volatile hydrocarbon-based oils such as, for example, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, isohexadecane, and for example, the oils sold under the trade names of Isopar or Permethyl, the C₈ to C₁₆ branched esters such as isohexyl or isodecyl neopentanoate, alcohols, and their mixtures.

Examples of volatile hydrocarbon-based oils include, but are not limited to those given in Table 1 below.

TABLE 1

| | Flash Point |
|---|---|
| Compound | (° C.) |
| Isododecane | 43 |
| Isohexadecane | 102 |
| Isodecyl neopentanoate | 118 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

The volatile solvent may also be chosen from volatile silicone oils, which may be linear or cyclic, having a viscosity, at room temperature, of less than or equal to 6 cSt, and having from 2 to 7 silicon atoms, optionally substituted with alkyl or alkoxy groups of 1 to 10 carbon atoms.

Examples of suitable volatile silicone oils include, but are not limited to, those listed in Table 2 below.

TABLE 2

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) | 56 | 1.5 |
| DC 200 (1.5 cSt) from Dow Corning | | |
| PDMS DC 200 (2 cSt) from Dow Corning | 87 | 2 |
| PDMS DC 200 (5 cSt) from Dow Corning | 134 | 5 |
| PDMS DC 200 (3 St) from Dow Corning | 102 | 3 |

The at least one volatile solvent is generally present in the cosmetic composition of the present invention in an amount ranging from about 5% to about 50% such as 3% or 5% to about 10%, 15%, 40% or 50%, all weights being based on the weight of the composition as a whole.

Perlite

Importantly, the compositions of the present invention also include perlite having an average (e.g., median) particle size of less than about 50 microns.

Perlite is a non-crystalline volcanic glass with a typical composition including 70-75% silicon dioxide, 12-15% aluminum oxide, 3-5% potassium oxide, 3-4% sodium oxide and traces of iron oxide, magnesium oxide and calcium oxide.

After mining, perlite is typically milled, dried and then calibrated in a first step. The product obtained, known as perlite ore, is gray-colored and is generally “expanded” using a high temperature process.

The expansion may be at about 1000° C. for several seconds where steam is forced out, When the temperature reaches 850-900° C., the water trapped in the structure of the material evaporates and brings about the expansion of the material, relative to its original volume. The expanded perlite particles in accordance with the invention may be obtained via the expansion process described in patent U.S. Pat. No. 5,002,698.

The expanded perlite particles used may be milled; in this case, they are known as Expanded Milled Perlite (EMP).

Perlites useful according to the invention have an average particle size (e.g., median diameter or “D50,” the size at which half the particles are larger and half are smaller) less than about 50 microns. According to certain embodiments, the perlite has an average particle size ranging from about 0.5 microns to about 50 microns, such as from about 10 microns to about 40 microns.

According to certain embodiments, the perlite particles have a loose bulk density at 25° C. ranging from 10 to 400 kg/m³ (standard DIN 53468) and preferably from 10 to 300 kg/m³.

According to certain embodiments, the perlite particles, the expanded perlite particles according to the invention have a water absorption capacity, measured at the wet point, ranging from 200% to 1500% and preferably from 250% to 800%.

The wet point corresponds to the amount of water which has to be added to 1 g of particle in order to obtain a homogeneous paste. This method is directly derived from the oil uptake method applied to solvents. The measurements are taken in the same manner by means of the wet point and the flow point, which have, respectively, the following definitions: [0080] wet point: weight expressed in grams per 100 g of product corresponding to the production of a homogeneous paste during the addition of a solvent to a powder; flow point: weight, expressed in grams per 100 g of product, at and above which the amount of solvent is greater than the ability of the powder to retain it. This is reflected by the production of a more or less homogeneous mixture which flows over the glass plate.

The wet point and the flow point are measured according to the following protocol using: a glass plate (25×25 mm), a spatula (wooden shaft and metal part, 15×2.7 mm); and a silk-bristled brush.

The glass plate is placed on the balance and 1 g of perlite particles is weighed out. The beaker containing the solvent and the liquid sampling pipette is placed on the balance. The solvent is gradually added to the powder, the whole being regularly blended (every 3 to 4 drops) by means of the spatula. The mass of solvent needed to obtain the wet point is noted. Further solvent is added and the mass which makes it possible to reach the flow point is noted. The mean of three tests will be taken.

According to certain embodiments, the expanded perlite has an oil absorption of at least about 70 g oil/100 g material, such as at least about 100 g oil/100 g material, such as from about 100 to about 300 g oil/100 g material, such as from about 150 to about 250 g oil/100 g material. The oil absorption may be determined using those familiar to one of ordinary skill in the art such as, for example, ASTM D281 (“Standard Test Method for Oil Absorption of Pigments by Spatula Rub-out”).

One notable filler pigment is perlite (e.g., expanded perlite). One notable perlite suitable for use in the composition is OPTIMAT 2550 available from Imerys S.A. The concentration of perlite in the composition is, according to certain embodiments at least about 10%, such as from about 10% to about 30%, such as from about 10% to about 20%, such as from about 10% to about 15% by weight.

The inventors have found that perlites having an average particle size less than about 50 microns when used in conjunction with the silicone resin and polyorganosiloxane copolymer film formers as well as the silicone elastomer and the volatile solvent, provide a surprising reduction of both tack and gloss without sacrificing long wear properties.

This can be particularly so when the expanded perlite is present at relatively high concentrations, such as from 10%, 11% or 12% to about 15%, 16%, 17%, 18%, 19% or 20% by weight. This can also be particularly so when the expanded perlite is present in concentrations relatively high compared to the silicone resin and polyorganosiloxane copolymer film formers. Accordingly, in certain embodiments, the expanded perlite, and the at least one silicone film former (e.g., at least one silicone resin and at least one polyorganosiloxane copolymer) are each present in a concentration by weight such that a ratio of the concentration by weight of the expanded perlite to a total concentration by weight of the at least one silicone film former is from about 0.3:1 or 0.5:1 to about 1:1, 1.5:1 or 2:1 In certain embodiments this ratio is from about 0.5:1 to about 1.5:1, such as from about 0.5:1 to about 1.1:1.

In certain other embodiments, the expanded perlite and the at least silicone elastomer are present in a ratio by weight such that a ratio of the concentration by weight of the expanded perlite to a total concentration by weight of the at least one silicone elastomer is from about 3:1 to about 30:1, such as from about 3:1 to about 10:1, such as from about such as from about 3:1 to about 10:1.

Non-Volatile Solvent

The compositions of the present invention also may include at least one non-volatile solvent (non-volatile oil).

The volatility of the oils can be determined using the evaporation speed as set forth in U.S. Pat. No. 6,338,839, the content of which is herein incorporated by reference.

Non-volatile oils include low viscosity oils (having a viscosity from about 5 to about 10 centipoise) and high viscosity oils (having a viscosity of from about 100 to about 10,000 centipoise), and mixtures thereof. In contrast to waxes, oils are liquids at room temperature.

According to a certain embodiment of the present invention, the oil is a high viscosity oil which is a silicone oil and/or a hydrocarbon oil. “High viscosity” means an oil having a viscosity greater than 100 cSt, particularly greater than 250 cSt at 25° C. Most particularly, the non-volatile oil is selected from a silicone oil. Such oils are described, for example in US 2011/0293550 and US 2004/0126350, both of which are herein incorporated by reference.

Non-limiting examples of suitable non-volatile silicone oils include polymethylsiloxanes (PDMS) with a linear or cyclic silicone chain, which are liquid or pasty at room temperature, especially cyclopolydimethylsiloxanes (cyclomethicones) such as cyclohexasiloxane; polydimethyl-siloxanes (CTFA designation “dimethicones”) comprising alkyl or alkoxy groups, which are pendent or at the end of a silicone chain, these groups containing from 2 to 24 carbon atoms; polydiethyl siloxanes; and dimethicone fluids such as dimethicone fluids having a viscosity in particular ranges of centipoise (cps) or centistokes (cSt) when measured at 25° C. For example, the viscosity may be from about 300 cps or cSt at 25° C. to about 1500 cps or cSt at 25° C., such as from about 300 cps or cSt at 25° C. to about 1200 cps or cSt at 25° C.

Specific examples of suitable for this invention high viscosity silicone oils include, but are not limited to, Xiameter® silicone fluids from Dow Corning.

According to certain embodiments, the composition is substantially free or devoid of non-volatile solvents having at least one or more phenyl groups. These solvents are for example described in U.S. Pat. No. 8,945,525, the entire content of which is hereby incorporated by the reference.

The at least one non-volatile silicone oil is present in the compositions of the present invention in an amount ranging from about 2% to about 30% by weight, such as from about 2%, 4% or 6% by weight to about 10%, 15%, 20% or 30%, based on the total weight of the composition, including all ranges and subranges within these ranges.

Pigments

The cosmetic compositions of the present invention may also contain at least one cosmetically acceptable colorant such as a pigment or dyestuff. Examples of suitable pigments include, but are not limited to, inorganic pigments, organic pigments, lakes, pearlescent pigments, iridescent or optically variable pigments, and mixtures thereof. A pigment should be understood to mean inorganic or organic, white or colored particles. Said pigments may optionally be surface-treated within the scope of the present invention but are not limited to treatments such as silicones, perfluorinated compounds, lecithin, and amino acids.

Representative examples of inorganic pigments useful in the present invention include those selected from the group consisting of rutile or anatase titanium dioxide, coded in the Color Index under the reference CI 77,891; black, yellow, red and brown iron oxides, coded under references CI 77,499, 77, 492 and, 77,491; manganese violet (CI 77,742); ultramarine blue (CI 77,007); chromium oxide (CI 77,288); chromium hydrate (CI 77,289); and ferric blue (CI 77,510) and mixtures thereof.

Representative examples of organic pigments and lakes useful in the present invention include, but are not limited to, D&C Red No. 19 (CI 45,170), D&C Red No. 9 (CI 15,585), D&C Red No. 21 (CI 45,380), D&C Orange No. 4 (CI 15,510), D&C Orange No. 5 (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430) and the dye or lakes based on cochineal carmine (CI 75,570) and mixtures thereof.

Representative examples of pearlescent pigments useful in the present invention include those selected from the group consisting of the white pearlescent pigments such as mica coated with titanium oxide, mica coated with titanium dioxide, bismuth oxychloride, titanium oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof.

The at least one pigment may be present in the compositions of the present invention in an amount ranging from about 1% to about 20% by weight, such as from about 1% 2%, 4% or 5% by weight to about 5%, 6%, 8% 10% or 20%, based on the total weight of the composition, including all ranges and subranges within these ranges.

According to certain embodiments, the expanded perlite is present in relatively high concentrations relative to the at least one pigment (for example, the total amount of inorganic pigments and lake pigments). For example, the expanded perlite and the at least one pigment are each present in a concentration by weight such that a ratio of the concentration by weight of the expanded perlite to a total concentration by weight of the at least one pigment is from about 0.5:1 to about 10:1, such as from about 1:1 to about 4:1, such as from about 1.5:1 to about 2.5:1.

Wax

The cosmetic compositions of the present invention optionally may contain at least one wax.

For the purposes of the present invention, a wax is a lipophilic fatty compound that is solid at room temperature (25° C.), has a reversible solid/liquid change of state (that is, the state of the material may change based on temperature), has a melting point greater than 45° C., preferably greater than 55° C., more preferably between about 65° C. to about 120° C., and has anisotropic crystal organization in the solid state. The melting point of the wax may be measured using a differential scanning calorimeter (DSC), for example the calorimeter sold under the name DSC 30 by Mettler. For waxes that are derived from petroleum, such as microcrystalline wax, the melting point may be measured according to the drop ASTM method, D-127.

The waxes are those generally used in cosmetics and dermatology. The waxes may be of natural origin, for instance beeswax, carnauba wax, candelilla wax, ouricoury wax, Japan wax, cork fiber wax or sugar cane wax, paraffin wax, lignite wax, microcrystalline waxes, lanolin wax, montan wax, ozokerites and hydrogenated oils, for instance hydrogenated jojoba oil.

The waxes also may be of synthetic origin, for instance polyethylene waxes derived from the polymerization of ethylene, waxes obtained by Fischer-Tropsch synthesis, esters of fatty acids and of glycerides that are solid at 40° C.

Waxes of synthetic origin are preferable as they are more uniform and provide greater reproducibility than waxes of natural origin. Moreover, the waxes are preferably not silicone waxes.

Particular waxes include polyethylene waxes, for example the product sold under the name Performalene 500-L Polyethylene (New Phase Technology), and polymethylene waxes, for instance the product sold under the name Cirebelle 303 (Sasol).

The cosmetic compositions of the present invention may contain at least one polypropylsilsesquioxane wax substituted with alkyl units having at least 30 carbons.

Polypropylsilsesquioxane waxes, in general, have been disclosed in patent publication WO2005/100444 and U.S. Pat. No. 8,586,013, the entire contents of which are hereby incorporated by reference.

It should be noted, however, that not all polypropylsilsesquioxane waxes yield stable colored cosmetic emulsion products. More particularly, it has been found that only those polypropylsilsesquioxane waxes substituted with alkyl units having at least 30 carbons are stable.

A particularly preferred polypropylsilsesquioxane wax for use in the present invention is a C30-45 ALKYLDIMETHYLSILYL POLYPROPYLSILSESQUIOXANE commercially available from DOW CORNING under the tradename SW-8005 C30 Resin Wax.

When present in the instant compositions, the at least one wax may be present in an amount ranging from about 0.01% to about 30% by weight, from about 0.02% to about 25%, typically from about 0.03% to 15% by weight, preferably from about 0.05% to about 5% by weight, including all ranges and subranges therebetween, all weights based on the weight of the composition as a whole.

Fillers

Fillers that may be used in the compositions of the invention include, for example, silica powder; talc; polyamide particles and especially those sold under the name Orgasol by the company Atochem; polyethylene powders; microspheres based on acrylic copolymers, such as those based on ethylene glycol dimethacrylate/lauryl methacrylate copolymer sold by the company Dow Corning under the name Polytrap; expanded powders such as hollow microspheres and especially the microspheres sold under the name Expancel by the company Kemanord Plast or under the name Micropearl F 80 ED by the company Matsumoto; powders of natural organic materials such as crosslinked or noncrosslinked corn starch, wheat starch or rice starch, such as the powders of starch crosslinked with octenyl succinate anhydride, sold under the name Dry-Flo by the company National Starch; silicone resin microbeads such as those sold under the name Tospearl by the company Toshiba Silicone; clays (bentone, laponite, saponite, etc.); and mixtures thereof.

In one notable embodiment, the other fillers present in the composition include a swellable clay. By “swellable clay” it is meant a clay material that is capable of swelling in water. An example of a swellable clay are smectite clays. The crystal structure of the smectite group, is an octahedral alumina sheet between two tetrahedral silica sheets. In one notable embodiment, the swellable clay is bentonite. Bentonite is a rock formed of highly colloidal and plastic clays composed mainly of montmorillonite, a clay mineral of the smectite group, and is produced by in situ devitrification of volcanic ash. In addition to montmorillonite, bentonite may contain feldspar, cristobalite, and crystalline quartz. Bentonite has an ability to form thixotrophic gels with water, an ability to absorb large quantities of water. Variations in interstitial water and exchangeable cations in the interlayer space affect the properties of bentonite and thus the commercial uses of the different types of bentonite.

One notable swellable clay suitable for use in the composition is BENTONE GEL ISD V, commercially available from Elementis Specialties, East Windsor, N.J. BENTONE GEL GTCC V is a dispersion of organically (disterammonium) modified hectorite in isododecane with added propylene carbonate.

The fillers (e.g., swellable clay) may be present in the compositions of the present invention in an amount ranging from about 1% to about 10% by weight, such as from about 0.1%, 0.5%, or 1% by weight to about 2%, 3%, 5% or 10%, based on the total weight of the composition, including all ranges and subranges within these ranges. Propylene carbonate, if present, may be present in the compositions of the present invention in an amount ranging from about 0.25% to about 2% by weight, or, for example, in an amount such that the ratio by weight of swellable clay to propylene carbonate is about 2:1 to about 5:1.

While in certain embodiments, certain fillers such as hydrophobic silica aerogels may be included. In certain other embodiments, the compositions are substantially free of hydrophobic silica aerogels.

Silica aerogels are porous materials obtained by replacing (by drying) the liquid component of a silica gel with air. They are generally synthesized via a sol-gel process in liquid medium and then dried, usually by extraction of a supercritical fluid, the one most commonly used being supercritical CO₂. This type of drying makes it possible to avoid shrinkage of the pores and of the material. The sol-gel process and the various drying processes are described in detail in Brinker C J., and Scherer G. W., Sol-Gel Science: New York: Academic Press, 1990. Silica aerogels, in general, have been disclosed in U.S. Pat. No. 9,320,689, the entire content of which is hereby incorporated by reference.

As hydrophobic silica aerogels that may be used in the invention, examples that may be mentioned include the aerogel sold under the name VM-2260 (INCI name Silica silylate), by the company Dow Corning, the particles of which have a mean size of about 1000 microns and a specific surface area per unit of mass ranging from 600 to 800 m²/g.

Mention may also be made of the aerogels sold by the company Cabot under the references AEROGEL TLD 201, AEROGEL OGD 201, AEROGEL TLD 203, ENOVA® AEROGEL MT 1100, ENOVA AEROGEL MT 1200.

Use will be made more particularly of the aerogel sold under the name VM-2270 (INCI name Silica silylate), by the company Dow Corning, the particles of which have a mean size ranging from 5-15 microns and a specific surface area per unit of mass ranging from 600 to 800 m²/g.

The silica aerogel particles if used can be used in the inventive compositions from 0.1% to about 8% by weight, preferably from about 0.1%, 0.2%. or 0.5% to about 0.5%, 1% 2% or 5% by weight, all weights based on the weight of the composition as a whole.

Additives

The compositions of the present invention may further comprise at least one cosmetically or dermatologically acceptable additive such as aa additional thickener, an additional film former, a plasticizer, an antioxidant, an essential oil, a botanical extract, a fragrance, a preserving agent, a fragrance, a pasty fatty substance, a neutralizing agent, and a polymer, and cosmetically active agents and/or dermatological active agents such as, for example, emollients, moisturizers, vitamins, essential fatty acids and medicaments.

As per this invention, the additives are incorporated from about 0.01%, 0.5% or 1% to about 1% z, 2% or 5% by weight.

The compositions of the present invention are useful as compositions for making up the skin, in particular the lips.

Compositions may be made by methods known to those skilled in the art, such as by charging a vessel with one or more solvents or oil and adding various ingredients and mixing. Pigments may be pre-ground into a suspension or slurry prior to adding.

### EXAMPLES

The present invention will be better understood from the examples which follow. The examples are intended to be nonrestrictive and explanatory only, with the scope of the invention defined by the claims.

Method of Preparation of Inventive Composition(s)

The mixture of pigment, isododecane and MQ resin was ground to create a pigment paste. The blend was processed using Disconti Mill until the paste passed the Hegman Gauge test (ASTM D1210-05). Then, the paste grind was added to the remaining ingredients. The mixture was heated to 80° C. and stirred, until a homogeneous liquid composition was obtained. After that, the inventive composition was cooled down to the room temperature and transferred to desired containers and/or applicators.

Evaluation of Inventive Compositions: Methods and Test Results

Compositions were tested for tackiness and gloss and wear. Each of the tested products was tested two times.

Initially a composition (Comparative Example 0) was prepared with 10% by weight of a perlite having a large average particle size of at least 200 um with no substantial oil absorption. The sample film was evaluated by drawdown (1 mil thickness) on a stainless steel plate and allow to dry for at least 1 hr. The resulting film had a rough and extremely gritty surface and was not further evaluated for gloss and wear.

Compositions (Comparative Example 1, Inventive Examples 1-3) were also prepared with 5%, 10%, 15% or 20% by weight of expanded perlite having a D50 (median) particle size of about 25 microns and an oil absorption of about 200 g oil/100 material to about 225 g oil/100 g material. The remainder of each composition included lake pigment, volatile solvent, silicone resin, polyorganosiloxane, copolymer, silicone elastomer, swellable clay and non volatile silicone oil. In order to compensate for lower levels of expanded perlite, the formulas were compensated with extra (q.$) isododecane. The sample with 20% expanded perlite had a reduced amount of swellable clay to mitigate the large increase in viscosity that otherwise would have been present.

Similarly, formulas were prepared in which the expanded perlite was replaced with Oryza Sativa rice starch having a particle size of about 2 to 8 microns—at concentrations by weight of 1%, 2% and 5% (Comparative Examples 2-4). Also replaced formulas were prepared in which the expanded perlite was replaced with silica (SUNSPHERES H51 from Asahi Glass) having a particle size of about 5 microns—at concentrations by weight of 2%, 5% and 10% (Comparative Examples 5-7). Each of these particulate materials had an oil absorption less than that of the expanded perlite having a D50 (median) particle size of about 25 microns. The remainder of each composition included pigment, volatile solvent, silicone resin, organopolyorganosiloxane copolymer, silicone elastomer, and non-volatile silicone oil. In order to compensate for lower levels of expanded perlite, the formulas were compensated with extra (q.$) isododecane.

Testing methods are described below.

Tack Testing

The films of each formula were deposited onto contrast cards using a 1 MIL drawdown bar and an Automatic Drawdown Machine. The films immediately analyzed using a Texture Analyzer equipped with a 2 inch stainless steel cylinder (TA-93). Tack force was measured after repeatedly applying 1500 g-force every 2 seconds. Then, the values of the tackiness were correlated to the comfort of wear of the tested products. Comparative Examples 2-7 were not measured but were qualitatively observed to be considerably higher than the inventive Examples. For those samples in which measurements were taken, the samples having tackiness having values higher than 250 grams force, were considered to be very uncomfortable to wear. The tack values between 50-249 gr/force, indicated medium comfort, and those with values of less than 50 gr/force were considered to be comfortable.

The results for Tack testing are shown below in Table 1.

TABLE 1

| | EXPANDED PERLITE | TACK |
|---|---|---|
| EXAMPLE | (WT. %) | (g) |
| Comp 1 | 5% expanded perlite | 314 |
| Example 1 | 10% expanded perlite | 238 |
| Example 2 | 15% expanded perlite | 201 |
| Example 3 | 20% expanded perlite | 78.8 |
| Comp 2 | 1% Rice starch | * |
| Comp 3 | 2% Rice starch | * |
| Comp 4 | 5% Rice starch | * |
| Comp 5 | 2% Silica | * |
| Comp 6 | 5% Silica | * |
| Comp 7 | 10% Silica | * |

* Not measured but observed to be “high”

The results indicate that the various concentrations of rice starch and silica as well as the 5% expanded perlite having an average particle size of less than about 50 microns exhibited unacceptably high tack, whereas the samples prepared with 10% 15% or 20% expanded perlite having an average particle size of less than about 50 microns had dramatically lower tack.

Wear Testing

The formula films were prepared by using a BYK 1 mil Birdbar to draw down on a stainless steel plate and let dry for 24 hr. After dried, the film was rubbed 15 times with a kim-wipe paper towel to assess the amount of product remaining on the stainless steel plate. If the film did not come off, the result was recorded as “Good,” otherwise the result was recorded as “Poor.”

The results for Wear testing are shown below in Table 2.

TABLE 2

| | EXPANDED PERLITE | |
|---|---|---|
| EXAMPLE | (WT. %) | WEAR |
| Comp 1 | 5% expanded perlite | Good |
| Example 1 | 10% expanded perlite | Good |
| Example 2 | 15% expanded perlite | Good |
| Example 3 | 20% expanded perlite | Poor |
| Comp 2 | 1% Rice starch | Good |
| Comp 3 | 2% Rice starch | Good |
| Comp 4 | 5% Rice starch | Good |
| Comp 5 | 2% Silica | Good |
| Comp 6 | 5% Silica | Good |
| Comp 7 | 10% Silica | Poor |

The results indicate that surprisingly high weight percentages of the particular expanded perlite below 20% by weight do not adversely affect wear.

Gloss Testing

The formula film were prepared by using a BYK 1 mil Birdbar to draw down on a stainless steel plate and let dry for 24 hr. After dried, the film gloss was analyzed by using BYK micro-TRI-gloss equipment to assess the gloss level among the formulas. The lower the gloss reading, the more matte the film is. Gloss readings below four indicate strong mattifying performance.

The results for Tack testing are shown below in Table 3.

TABLE 3

| | EXPANDED PERLITE | |
|---|---|---|
| EXAMPLE | (WT. %) | Gloss |
| Comp 1 | 5% expanded perlite | 2.7 |
| Example 1 | 10% expanded perlite | 2.1 |
| Example 2 | 15% expanded perlite | 1.3 |
| Example 3 | 20% expanded perlite | 1.8 |
| Comp 2 | 1% Rice starch | 7.1 |
| Comp 3 | 2% Rice starch | 6.6 |
| Comp 4 | 5% Rice starch | 4.2 |
| Comp 5 | 2% Silica | 15.9 |
| Comp 6 | 5% Silica | 11.7 |
| Comp 7 | 10% Silica | 5 |

The results indicate that expanded perlite reduces gloss in these compositions to provide strong mattifying performance and generally increasing the level of expanded perlite reduces gloss. This can be highly desirable for matte-finish lip products.

What is claimed is:

1. A cosmetic composition comprising:
at least one silicone film former;
from about 10% by weight to about 20% by weight of expanded perlite having an average particle size of less than about 50 microns;
at least one silicone elastomer; and
at least one volatile solvent, wherein the composition is anhydrous.

2. The cosmetic composition of claim 1, wherein the expanded perlite, the at least one silicone film-former are each present in a concentration by weight such that a ratio of the concentration by weight of the expanded perlite to a total concentration by weight of the at least one silicone film-former is from about 0.5:1 to about 1.5:1.

3. The cosmetic composition of claim 2, wherein the at least one silicone film-former includes at least one silicone resin and at least one polyorganosiloxane copolymer.

4. The cosmetic composition of claim 1 further comprising at least one colorant.

5. The cosmetic composition of claim 4 wherein the expanded perlite and the at least one colorant are both present in a concentration by weight such that a ratio of the concentration by weight of the expanded perlite to the concentration by weight of the at least one colorant is from about 1:1 to about 4:1.

6. The cosmetic composition of claim 5 wherein the at least one silicone film-former includes at least one silicone resin and at least one polyorganosiloxane copolymer and the at least one silicone resin and the at least one polyorganosiloxane copolymer are each present in a concentration by weight such that a ratio of the concentration by weight of the expanded perlite to a total concentration by weight of the at least one silicone resin and the at least one polyorganosiloxane copolymer is from about 0.5:1 to about 1.1:1.

7. The cosmetic composition of claim 3 wherein the at least one silicone resin is present in a concentration by weight from about 4% to about 25%.

8. The cosmetic composition of claim 3 wherein the at least one polyorganosiloxane copolymer is present in a concentration by weight from about 1% to about 25%.

9. The cosmetic composition of claim 1 wherein the at least one silicone elastomer is present in a concentration by weight from about 1% to about 20%.

10. The cosmetic composition of claim 1 wherein the at least one volatile solvent is present in a concentration by weight from about 5% or about 50%.

11. The cosmetic composition of claim 1, wherein the at least one silicone elastomer is non-emulsifying silicone elastomer.

12. The cosmetic composition of claim 1, wherein the at least one silicone elastomer is a dimethicone crosspolymer and is present in the cosmetic composition in a concentration by weight from about 1% to about 10%.

13. The cosmetic composition of claim 1, wherein the volatile solvent is selected from hydrocarbon oils, silicone oils and mixtures thereof.

14. The cosmetic composition of claim 1, further comprising at least one non-volatile oil, wherein the non-volatile oil is dimethicone fluid having a viscosity greater than about 300 cSt and lower than about 1200 cSt at 25° C.

15. The cosmetic composition of claim 14, wherein the at least one dimethicone fluid is present in concentration by weight from about 6% to about 20% by weight being relative to the total weight of the composition.

16. The cosmetic composition of claim 3, wherein the at least one polyorganosiloxane copolymer is Nylon-611/Dimethicone copolymer, and is present in concentration by weight from about 7% to about 20% by weight being relative to the total weight of the composition.

17. The composition of claim 1, wherein the expanded perlite has an oil absorption value of at least about 100 grams of oil per 100 grams of expanded perlite.

18. The cosmetic composition of claim 1 wherein the composition is substantially free of hydrophobic silicas.

19. An anhydrous lipstick liquid composition comprising:
from about 4% to about 25% by weight of at least one silicone resin;
from about 1% to about 25% by weight of at least one polyorganosiloxane copolymer;
from about 10% by weight to about 20% by weight of expanded perlite having an average particle size of less than about 50 microns;
from about 1% to about 25% by weight of least one silicone elastomer;
from about 0.5% to about 5% of swellable clay;
from about 5% to about 50% by weight from at least one volatile solvent; and
from about 6% to about 20% by weight of a dimethicone fluid having a viscosity greater than about 300 cSt and lower than about 1200 cSt at 25° C.

20. A method of making up lips comprising applying to the lips a lipstick composition comprising:
at least one silicone film former;
from about 10% by weight to about 20% by weight of expanded perlite having an average particle size of less than about 50 microns;
at least one silicone elastomer; and
at least one volatile solvent, wherein the composition is anhydrous.
